(19) United States
(12) Patent Application Publication
(10) Pub. No.: US 2023/0391746 A1
(43) Pub. Date: Dec. 7, 2023

(54) SOLID STATE FORMS OF PRALSETINIB AND PROCESS FOR PREPARATION THEREOF

(71) Applicant: TEVA PHARMACEUTICALS INTERNATIONAL GMBH, Jona (CH)

(72) Inventors: Dijana Skalec Samec, Jastrebarsko (HR); Ivana Landeka, Zagreb (HR)

(73) Assignee: TEVA PHARMACEUTICALS INTERNATIONAL GMBH, Jona (CH)

(21) Appl. No.: 18/032,208

(22) PCT Filed: Oct. 19, 2021
(86) PCT No.: PCT/US21/55516

§ 371 Date: Apr. 17, 2023

Related U.S. Application Data
(60) Provisional application No. 63/181,374, filed on Apr. 29, 2021.
(60) Provisional application No. 63/128,226, filed on Dec. 21, 2020.
(60) Provisional application No. 63/111,125, filed on Nov. 9, 2020.
(60) Provisional application No. 63/093,340, filed on Oct. 19, 2020.

(51) Int. Cl. C07D 401/14

(52) U.S. Cl. CPC C07D 401/14; C07B 2200/13

(57) ABSTRACT

The present disclosure encompasses solid state forms of Pralsetinib and of Pralsetinib salts and co-crystals, in embodiments crystalline polymorphs of Pralsetinib, Pralsetinib salts and co-crystals processes for preparation thereof, and pharmaceutical compositions thereof. The present disclosure encompasses solid state forms of Pralsetinib and of Pralsetinib salts and co-crystals, in embodiments crystalline polymorphs of Pralsetinib, Pralsetinib salts and co-crystals processes for preparation thereof, and pharmaceutical compositions thereof.

### FIELD OF THE DISCLOSURE

The present disclosure encompasses solid state forms of Pralsetinib and of Pralsetinib salts and co-crystals, in embodiments crystalline polymorphs of Pralsetinib, Pralsetinib salts and co-crystals processes for preparation thereof, and pharmaceutical compositions thereof.

### BACKGROUND OF THE DISCLOSURE

Pralsetinib, (cis)-N—((S)-1-(6-(4-fluoro-1H-pyrazol-1-yl)pyridin-3-yl)ethyl)-1-methoxy-4-(4-methyl-6-(5-methyl-1H-pyrazol-3-ylamino)pyrimidin-2-yl)cyclohexanecarboxamide, has the following chemical structure:

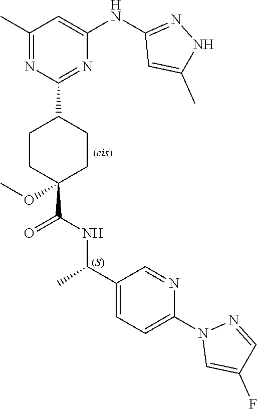

Pralsetinib is an oral receptor tyrosine kinase inhibitor, and it is approved for the treatment of adult patients with metastatic RET fusion-positive non-small cell lung cancer (NSCLC) and is developed for treatment of RET-mutant medullary thyroid cancer and RET fusion-positive thyroid cancer. Pralsetinib is also under clinical investigation for the treatment of other RET-altered solid tumors.

The compound is described in U.S. Pat. No. 10,030,005.

CN 111777595 discloses crystalline forms of Pralsetinib.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Pralsetinib.

### SUMMARY OF THE DISCLOSURE

The present disclosure provides crystalline polymorphs of Pralsetinib, and of Pralsetinib salts and co-crystals, processes for preparation thereof, and pharmaceutical compositions thereof. These crystalline polymorphs can be used to prepare other solid state forms of Pralsetinib, Pralsetinib salts and their solid state forms.

The present disclosure also provides uses of the said solid state forms of Pralsetinib, Pralsetinib salts and co-crystals in the preparation of other solid state forms of Pralsetinib or salts thereof.

The present disclosure provides crystalline polymorphs of Pralsetinib, and of Pralsetinib salts and co-crystals for use in medicine, including for the treatment of non-small cell lung cancer (NSCLC) and thyroid cancer, or other RET-altered solid tumors. The present disclosure also encompasses the use of crystalline polymorphs of Pralsetinib of the present disclosure for the preparation of pharmaceutical compositions and/or formulations.

In another aspect, the present disclosure provides pharmaceutical compositions comprising crystalline polymorphs of Pralsetinib and of Pralsetinib salts and co-crystals according to the present disclosure.

The present disclosure includes processes for preparing the above mentioned pharmaceutical compositions. The processes include combining any one or a combination of the crystalline polymorphs of Pralsetinib or of Pralsetinib salts and co-crystals with at least one pharmaceutically acceptable excipient.

The crystalline polymorphs of Pralsetinib and of Pralsetinib salts and co-crystals as defined herein and the pharmaceutical compositions or formulations of the crystalline polymorph of Pralsetinib may be used as medicaments, such as non-small cell lung cancer (NSCLC) and thyroid cancer, or other RET-altered solid tumors.

The present disclosure also provides methods of treating non-small cell lung cancer (NSCLC) and thyroid cancer, or other RET-altered solid tumors, by administering a therapeutically effective amount of any one or a combination of the crystalline polymorphs of Pralsetinib of the present disclosure, or at least one of the above pharmaceutical compositions, to a subject suffering from non-small cell lung cancer (NSCLC) and thyroid cancer, or other RET-altered solid tumors, or otherwise in need of the treatment.

The present disclosure also provides uses of crystalline polymorphs of Pralsetinib of the present disclosure, or at least one of the above pharmaceutical compositions, for the manufacture of medicaments for treating e.g., non-small cell lung cancer (NSCLC) and thyroid cancer, or other RET-altered solid tumors.

### BRIEF DESCRIPTION OF THE DRAWINGS

FIG. 1 shows a characteristic X-ray powder diffraction pattern (XRPD) of Pralsetinib Form II;

FIG. 2 shows a characteristic X-ray powder diffraction pattern (XRPD) of Pralsetinib Form III;

FIG. 3 shows a characteristic X-ray powder diffraction pattern (XRPD) of Pralsetinib Form V;

FIG. 4 shows a characteristic X-ray powder diffraction pattern (XRPD) of Pralsetinib Form VI;

FIG. 5 shows a characteristic X-ray powder diffraction pattern (XRPD) of Pralsetinib Form IV;

FIG. 6 shows a characteristic X-ray powder diffraction pattern (XRPD) of Pralsetinib Form VII;

FIG. 7 shows a characteristic X-ray powder diffraction pattern (XRPD) of Pralsetinib Form VIII;

FIG. 8 shows a characteristic X-ray powder diffraction pattern (XRPD) of Pralsetinib Form IX;

FIG. 9 shows a characteristic X-ray powder diffraction pattern (XRPD) of Pralsetinib hydrochloride Form I;

FIG. 10 shows a characteristic X-ray powder diffraction pattern (XRPD) of Pralsetinib Form X;

FIG. 11 shows a characteristic X-ray powder diffraction pattern (XRPD) of Pralsetinib Form XI;

FIG. 12 shows a characteristic X-ray powder diffraction pattern (XRPD) of Pralsetinib Form XII;

FIG. 13 shows a characteristic X-ray powder diffraction pattern (XRPD) of Pralsetinib Form XIII;

FIG. 14 shows a characteristic X-ray powder diffraction pattern (XRPD) of Pralsetinib L-(+)-tartrate Form I;

FIG. 15 shows a characteristic X-ray powder diffraction pattern (XRPD) of Pralsetinib tosylate Form I;

FIG. 16 shows a characteristic X-ray powder diffraction pattern (XRPD) of Pralsetinib: benzoic acid Form I;

FIG. 17 shows a characteristic X-ray powder diffraction pattern (XRPD) of Pralsetinib hydrochloride Form II;

FIG. 18 shows a characteristic solid state ¹³C NMR spectrum of Pralsetinib Form V;

FIG. 19 shows a characteristic solid state ¹³C NMR spectrum of Pralsetinib Form IX;

FIG. 20 shows a characteristic solid state ¹³C NMR spectrum of Pralsetinib Form XI; and

FIG. 21 shows a characteristic X-ray powder diffraction pattern (XRPD) of Pralsetinib Form V, obtained according to example 19.

### DETAILED DESCRIPTION OF THE DISCLOSURE

The present disclosure encompasses solid state forms of Pralsetinib, and of Pralsetinib salts and co-crystals including crystalline polymorphs of Pralsetinib, crystalline polymorphs of Pralsetinib salts and co-crystals, processes for preparation thereof, and pharmaceutical compositions thereof.

Solid state properties of Pralsetinib, Pralsetinib salts and co-crystals and crystalline polymorphs thereof can be influenced by controlling the conditions under which Pralsetinib and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Pralsetinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Pralsetinib. In some embodiments of the disclosure, the described crystalline polymorph of Pralsetinib may contain from about 1% to about 20% (w/w), from about 5% to about 20% (w/w), or from about 5% to about 10% (w/w) of one or more other crystalline polymorph of the same Pralsetinib.

Depending on which other crystalline polymorphs a comparison is made, the crystalline polymorphs of Pralsetinib of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Pralsetinib referred to herein as being characterized by graphical data “as depicted in” or “as substantially depicted in” a Figure will thus be understood to include any crystal forms of Pralsetinib characterized with the graphical data having such small variations, as are well known to the skilled person, in comparison with the Figure.

“Co-Crystal” or “Co-crystal” as used herein is defined as a crystalline material including two or more molecules in the same crystalline lattice and associated by non-ionic and non-covalent bonds. In some embodiments, the co-crystal includes two molecules which are in natural state. In embodiments the molar ratio between the active pharmaceutical ingredient (Pralsetinib) and the coformer (benzoic acid) is between 1:1.5 and 1.5:1, preferably between 1:1.25 and 1.25:1, in other embodiments about 1:1.

The solid state form may be referred to herein as “Pralsetinib Form name” or “Crystalline Form name of Pralsetinib” or “Crystalline Pralsetinib Form name” or “Crystalline polymorph name of Pralsetinib” or “Crystalline Pralsetinib polymorph name” or “Pralsetinib polymorph name”. For example, crystalline form V of Pralsetinib may be interchangeably referred to herein as Pralsetinib Form V or as Crystalline Pralsetinib Form V or as Crystalline polymorph V of Pralsetinib or as Crystalline Pralsetinib polymorph V or Pralsetinib polymorph V.

As used herein, and unless stated otherwise, the term “anhydrous” in relation to crystalline forms of Pralsetinib, relates to a crystalline form of Pralsetinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Pralsetinib of the present disclosure corresponds to a crystalline polymorph of Pralsetinib that is physically separated from the reaction mixture in which it is formed.

As used herein, unless stated otherwise, the XRPD measurements are taken using copper Kα radiation wavelength 1.54184 Å. XRPD peaks reported herein are measured using CuK α radiation, λ=1.54184 Å, typically at a temperature of 25±3° C.

As used herein, unless stated otherwise, ¹³C NMR reported herein are measured at 125 MHz at a magic angle spinning frequency ωᵣ/27c=11 kHz, preferably at a temperature of at 293 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

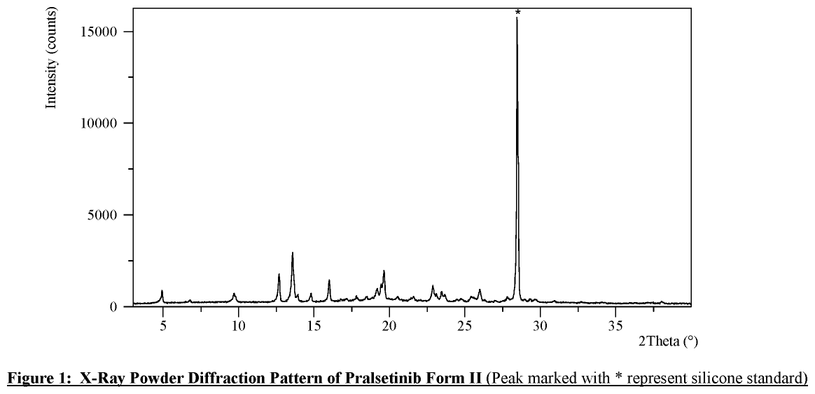

The present disclosure includes a crystalline polymorph of Pralsetinib, designated Form II. The crystalline Form II of Pralsetinib may be characterized by data selected from one or more of the following: an X-ray powder diffraction pattern substantially as depicted in FIG. 1; an X-ray powder diffraction pattern having peaks at 4.9, 9.7, 12.7, 14.8 and 16.0 degrees 2-theta±0.2 degrees 2-theta; and combinations of these data.

Crystalline Form II of Pralsetinib may be further characterized by an X-ray powder diffraction pattern having peaks at 4.9, 9.7, 12.7, 14.8 and 16.0 degrees 2-theta±0.2 degrees 2-theta, and also having any one, two, three, or four additional peaks selected from 6.8, 17.8, 19.6 and 22.9 degrees 2-theta±0.2 degrees 2-theta.

According to any aspect or embodiment of the present disclosure, crystalline Form II of Pralsetinib may be alternatively characterized by an X-ray powder diffraction pattern having peaks at 4.9, 6.8, 9.7, 12.7, 14.8, 16.0, 17.8, 19.6, and 22.9 degrees 2-theta±0.2 degrees 2-theta.

In one embodiment of the present disclosure, crystalline Form II of Pralsetinib is isolated.

Crystalline Form II of Pralsetinib may be characterized by each of the above characteristics alone or by all possible combinations, e.g., an XRPD pattern having peaks at 4.9, 9.7, 12.7, 14.8 and 16.0 degrees 2-theta±0.2 degrees 2-theta; an XRPD pattern as depicted in FIG. 1, and combinations thereof.

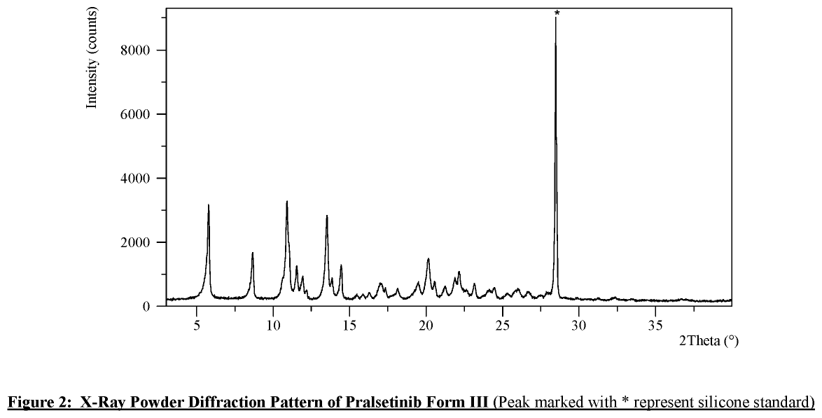

The present disclosure includes a crystalline polymorph of Pralsetinib, designated Form III. The crystalline Form III of Pralsetinib may be characterized by data selected from one or more of the following: an X-ray powder diffraction pattern substantially as depicted in FIG. 2; an X-ray powder diffraction pattern having peaks at 10.9, 11.9, 13.5, 16.3 and 20.2 degrees 2-theta±0.2 degrees 2-theta; and combinations of these data.

Crystalline Form III of Pralsetinib may be further characterized by an X-ray powder diffraction pattern having peaks at 10.9, 11.9, 13.5, 16.3 and 20.2 degrees 2-theta±0.2 degrees 2-theta, and also having any one, two, three, or four additional peaks selected from 12.2, 19.5, 21.2 and 23.2 degrees 2-theta±0.2 degrees 2-theta.

According to any aspect or embodiment of the present disclosure, crystalline Form III of Pralsetinib may be alternatively characterized by an X-ray powder diffraction pattern having peaks at 10.9, 11.9, 12.2, 13.5, 16.3, 19.5, 20.2, 21.2, and 23.2 degrees 2-theta±0.2 degrees 2-theta.

In one embodiment of the present disclosure, crystalline Form III of Pralsetinib is isolated.

Crystalline Form III of Pralsetinib may be characterized by each of the above characteristics alone or by all possible combinations, e.g., an XRPD pattern having peaks at 10.9, 11.9, 13.5, 16.3 and 20.2 degrees 2-theta±0.2 degrees 2-theta; an XRPD pattern as depicted in FIG. 2, and combinations thereof.

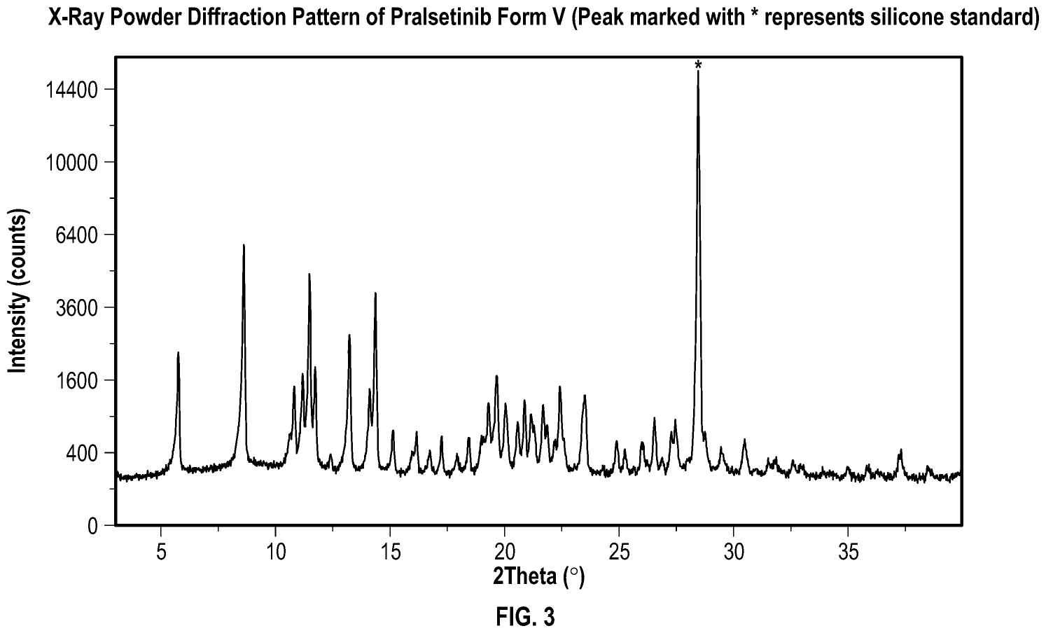

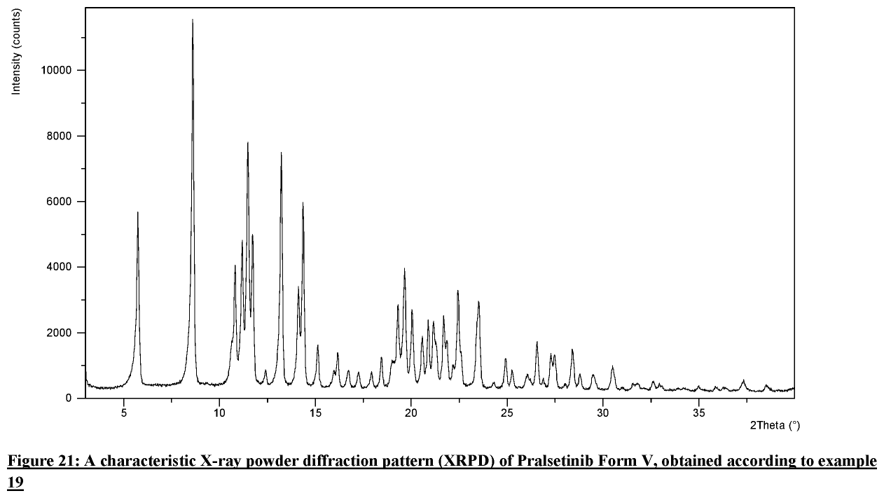

The present disclosure includes a crystalline polymorph of Pralsetinib, designated Form V. The crystalline Form V of Pralsetinib may be characterized by data selected from one or more of the following: an X-ray powder diffraction pattern substantially as depicted in FIG. 3 or FIG. 21; an X-ray powder diffraction pattern having peaks at 11.2, 11.7, 13.2, 15.1 and 17.2 degrees 2-theta±0.2 degrees 2-theta; and combinations of these data.

Crystalline Form V of Pralsetinib may be further characterized by an X-ray powder diffraction pattern having peaks at 11.2, 11.7, 13.2, 15.1 and 17.2 degrees 2-theta±0.2 degrees 2-theta, and also having any one, two, three, four, or five additional peaks selected from 14.1, 19.7, 20.9, 22.4 and 23.5 degrees 2-theta±0.2 degrees 2-theta.

According to any aspect or embodiment of the present disclosure, crystalline Form V of Pralsetinib may be alternatively characterized by an X-ray powder diffraction pattern having peaks at 11.2, 11.7, 13.2, 14.1, 15.1, 17.2, 19.7, 20.9, 22.4, and 23.5 degrees 2-theta±0.2 degrees 2-theta.

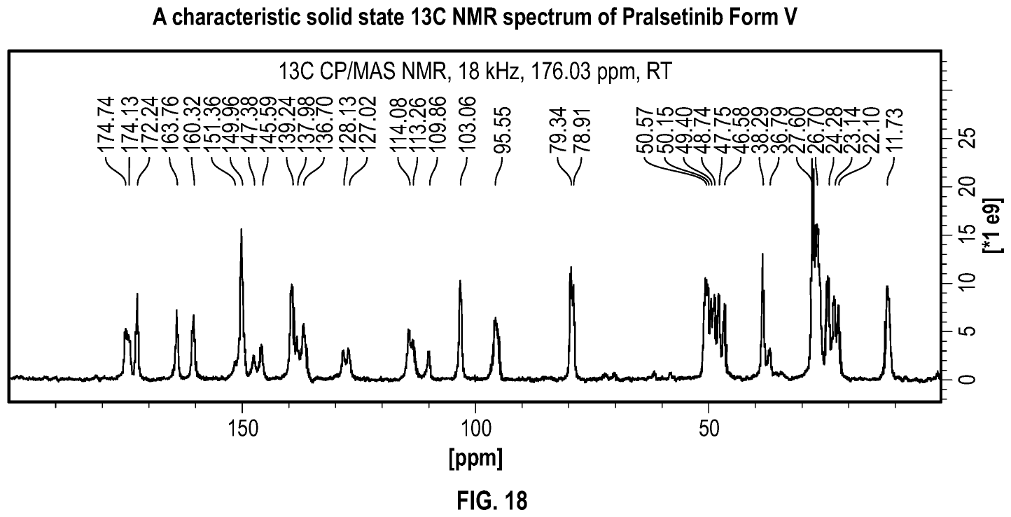

Alternatively or additionally, according to any aspect or embodiment of the present disclosure, Crystalline Form V of Pralsetinib may be characterized by data selected from one or more of the following: a solid state ¹³C NMR having peaks at 172.2, 160.3, 103.1 and 38.3 ppm±0.2 ppm; or a solid state ¹³C NMR spectrum having chemical shift differences between a reference peak at 11.7±0.2 ppm of: 160.5, 148.6, 91.3 and 26.6±0.1 ppm respectively; or by a solid state ¹³C NMR spectrum substantially as depicted in FIG. 18; and combinations of these data.

According to any aspect or embodiment of the present disclosure, crystalline Form V of Pralsetinib may be characterized by any of the above, and in addition, an X-ray powder diffraction pattern having an absence of peaks at 6.2 to 8.1 degrees 2-theta±0.2 degrees 2-theta.

According to any aspect or embodiment of the present disclosure, crystalline Form V of Pralsetinib may be characterized by any of the above, and in addition, an X-ray powder diffraction pattern having an absence of peaks at 9.0 to 10.2 degrees 2-theta±0.2 degrees 2-theta.

According to any aspect or embodiment of the present disclosure, crystalline Form V of Pralsetinib may be characterized by any of the above, and in addition, an X-ray powder diffraction pattern having an absence of peaks at 6.2 to 8.1 degrees 2-theta and an absence of peaks at 9.0 to 10.2 degrees 2-theta±0.2 degrees 2-theta.

Crystalline Form V of Pralsetinib may be alternatively characterised by the following unit cell data:

| Unit cell parameter | Le Bail |
|---|---|
| cell_length_a | 8.3649(8) Å |
| cell_length_b | 5.6382 (7) Å |
| cell_length_c | 30.9041 (4) Å |
| cell_angle_alpha | 90° |
| cell_angle_beta | 93.644(6)° |
| cell_angle_gamma | 90° |
| cell_volume | 1454.6 (3) Å³ |
| symmetry_cell_setting | Monoclinic |
| Symmetry space group name | P2₁ |

In one embodiment of the present disclosure, crystalline Form V of Pralsetinib is isolated.

Crystalline Form V of Pralsetinib may be characterized by each of the above characteristics alone or by all possible combinations, e.g., an XRPD pattern having any of the characteristic peaks set out above, and the unit cell data set out above, or an XRPD pattern having peaks at 11.2, 11.7, 13.2, 15.1 and 17.2 degrees 2-theta±0.2 degrees 2-theta; an XRPD pattern as depicted in FIG. 3, or a solid state ¹³C NMR spectrum substantially as depicted in FIG. 18 and any other combinations thereof.

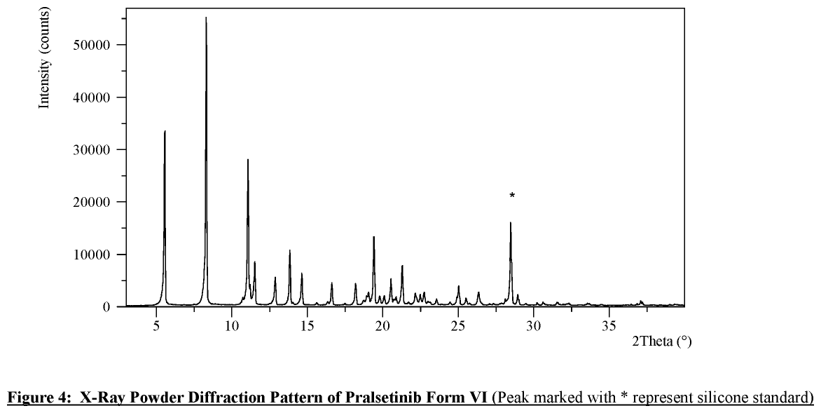

The present disclosure includes a crystalline polymorph of Pralsetinib, designated Form VI. The crystalline Form VI of Pralsetinib may be characterized by data selected from one or more of the following: an X-ray powder diffraction pattern substantially as depicted in FIG. 4; an X-ray powder diffraction pattern having peaks at 5.5, 7.3, 12.9, 14.6 and 16.6 degrees 2-theta±0.2 degrees 2-theta; and combinations of these data.

Crystalline Form VI of Pralsetinib may be further characterized by an X-ray powder diffraction pattern having peaks at 5.5, 7.3, 12.9, 14.6 and 16.6 degrees 2-theta±0.2 degrees 2-theta, and also having any one, two, three, four or five additional peaks selected from 11.1, 13.8, 19.4, 20.5 and 25.0 degrees 2-theta±0.2 degrees 2-theta.

According to any aspect or embodiment of the present disclosure, crystalline Form VI of Pralsetinib may be alternatively characterized by an X-ray powder diffraction pattern having peaks at 5.5, 7.3, 11.1, 12.9, 13.8, 14.6, 16.6, 19.4, 20.5, and 25.0 degrees 2-theta±0.2 degrees 2-theta.

In one embodiment of the present disclosure, crystalline Form VI of Pralsetinib is isolated.

Crystalline Form VI of Pralsetinib may be characterized by each of the above characteristics alone or by all possible combinations, e.g., an XRPD pattern having peaks at 7.3, 12.9, 14.6 and 16.6 degrees 2-theta±0.2 degrees 2-theta; an XRPD pattern as depicted in FIG. 4, and combinations thereof.

The present disclosure includes a crystalline polymorph of Pralsetinib, designated Form IV. The crystalline Form IV of Pralsetinib may be characterized by data selected from one or more of the following: an X-ray powder diffraction pattern substantially as depicted in Figure an X-ray powder diffraction pattern having peaks at 9.3, 10.4, 11.3, 17.6 and 18.2 degrees 2-theta±0.2 degrees 2-theta; and combinations of these data.

Crystalline Form IV of Pralsetinib may be further characterized by an X-ray powder diffraction pattern having peaks at 9.3, 10.4, 11.3, 17.6 and 18.2 degrees 2-theta±0.2 degrees 2-theta, and also having any one, two, three, four or five additional peaks selected from 14.0, 18.5, 21.1, 21.9 and 23.9 degrees 2-theta±0.2 degrees 2-theta.

According to any aspect or embodiment of the present disclosure, crystalline Form IV of Pralsetinib may be alternatively characterized by an X-ray powder diffraction pattern having peaks at 9.3, 10.4, 11.3, 14.0, 17.6, 18.2, 18.5, 21.1, 21.9, and 23.9 degrees 2-theta±0.2 degrees 2-theta.

In one embodiment of the present disclosure, crystalline Form IV of Pralsetinib is isolated.

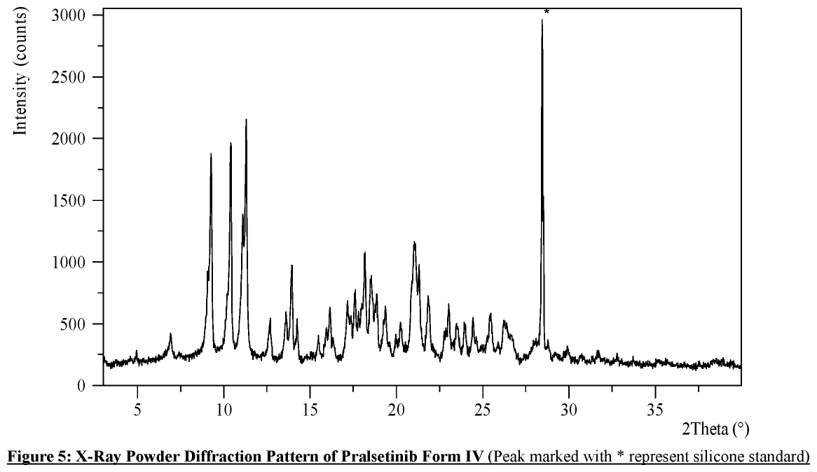

Crystalline Form IV of Pralsetinib may be characterized by each of the above characteristics alone or by all possible combinations, e.g., an XRPD pattern having peaks at 9.3, 10.4, 11.3, 17.6 and 18.2 degrees 2-theta±0.2 degrees 2-theta; an XRPD pattern as depicted in FIG. 5, and combinations thereof.

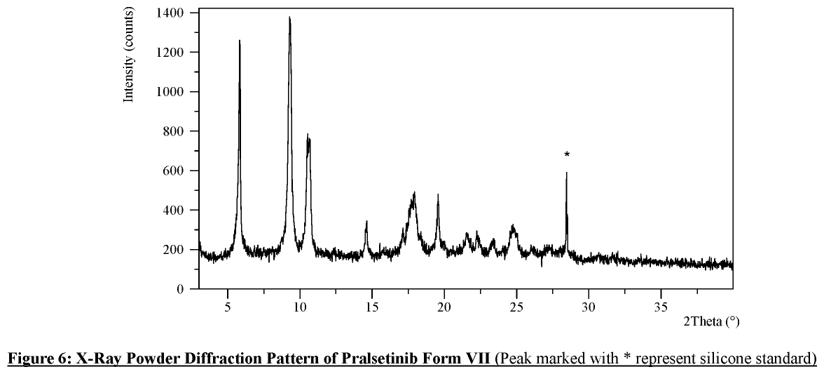

The present disclosure includes a crystalline polymorph of Pralsetinib, designated Form VII. The crystalline Form VII of Pralsetinib may be characterized by data selected from one or more of the following: an X-ray powder diffraction pattern substantially as depicted in FIG. 6; an X-ray powder diffraction pattern having peaks at 5.8, 9.3, 10.5, 14.6 and 19.6 degrees 2-theta±0.2 degrees 2-theta; an X-ray powder diffraction pattern having peaks at 5.8, 9.4, 10.5, 14.6 and 19.6 degrees 2-theta±0.2 degrees 2-theta and combinations of these data.

Crystalline Form VII of Pralsetinib may be further characterized by an X-ray powder diffraction pattern having peaks at 5.8, 9.3, 10.5, 14.6 and 19.6 degrees 2-theta±0.2 degrees 2-theta, or an X-ray powder diffraction pattern having peaks at 5.8, 9.4, 10.5, 14.6 and 19.6 degrees 2-theta±0.2 degrees 2-theta, and also having any one or two additional peaks selected from 17.9 and 22.3 degrees 2-theta±0.2 degrees 2-theta; or from 17.9 and 22.2 degrees 2-theta±degrees 2-theta.

Crystalline Form VII of Pralsetinib may be alternatively characterized by an X-ray powder diffraction pattern having peaks at 5.8, 9.3, 10.5, 14.6, 17.9, 19.6, and 22.3 degrees 2-theta±0.2 degrees 2-theta

Crystalline Form VII of Pralsetinib may be alternatively characterized by an X-ray powder diffraction pattern having peaks at 5.8, 9.3, 10.5, 14.6, 17.9, 19.6, and 22.2 degrees 2-theta±0.2 degrees 2-theta.

Crystalline Form VII of Pralsetinib may be alternatively characterized by an X-ray powder diffraction pattern having peaks at 5.8, 9.4, 10.5, 14.6, 17.9, 19.6, and 22.3 degrees 2-theta±0.2 degrees 2-theta

Crystalline Form VII of Pralsetinib may be alternatively characterized by an X-ray powder diffraction pattern having peaks at 5.8, 9.4, 10.5, 14.6, 17.9, 19.6, and 22.2 degrees 2-theta±0.2 degrees 2-theta.

In one embodiment of the present disclosure, crystalline Form VII of Pralsetinib is isolated.

Crystalline Form VII of Pralsetinib may be characterized by each of the above characteristics alone or by all possible combinations, e.g., an XRPD pattern having peaks at 9.3, 10.5, 14.6 and 19.6 degrees 2-theta±0.2 degrees 2-theta; an XRPD pattern as depicted in FIG. 6, and combinations thereof.

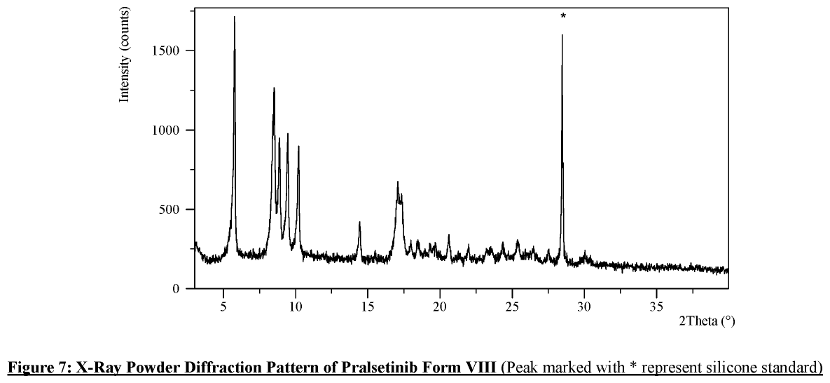

The present disclosure includes a crystalline polymorph of Pralsetinib, designated Form VIII. The crystalline Form VIII of Pralsetinib may be characterized by data selected from one or more of the following: an X-ray powder diffraction pattern substantially as depicted in FIG. 7; an X-ray powder diffraction pattern having peaks at 8.5, 8.9, 9.5, 10.2 and 14.4 degrees 2-theta±0.2 degrees 2-theta; and combinations of these data.

Crystalline Form VIII of Pralsetinib may be further characterized by an X-ray powder diffraction pattern having peaks at 8.5, 8.9, 9.5, 10.2 and 14.4 degrees 2-theta±0.2 degrees 2-theta, and also having any one or two additional peaks selected from 17.1 and 20.6 degrees 2-theta±0.2 degrees 2-theta.

According to any aspect or embodiment of the present disclosure, crystalline Form VIII of Pralsetinib may be alternatively characterized by an X-ray powder diffraction pattern having peaks at 8.5, 8.9, 9.5, 10.2, 14.4, 17.1, and 20.6 degrees 2-theta±0.2 degrees 2-theta.

In one embodiment of the present disclosure, crystalline Form VIII of Pralsetinib is isolated.

Crystalline Form VIII of Pralsetinib may be characterized by each of the above characteristics alone or by all possible combinations, e.g., an XRPD pattern having peaks at 8.5, 8.9, 9.5, 10.2 and 14.4 degrees 2-theta±0.2 degrees 2-theta; an XRPD pattern as depicted in FIG. 7, and combinations thereof.

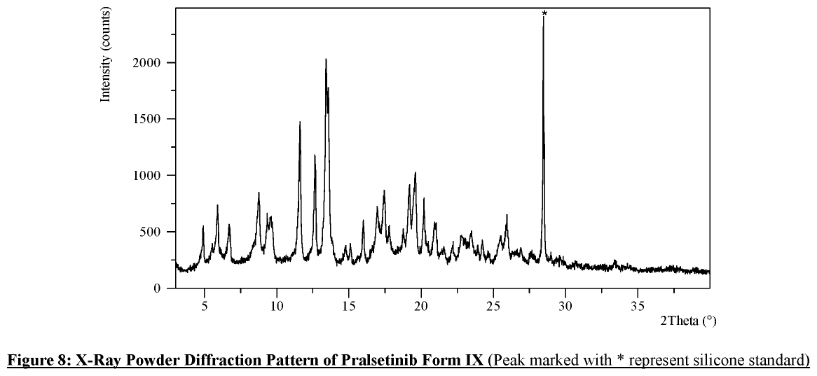

The present disclosure includes a crystalline polymorph of Pralsetinib, designated Form IX. The crystalline Form IX of Pralsetinib may be characterized by data selected from one or more of the following: an X-ray powder diffraction pattern substantially as depicted in FIG. 8; an X-ray powder diffraction pattern having peaks at 6.7, 9.7, 11.6, 13.4 and 17.0 degrees 2-theta±0.2 degrees 2-theta; and combinations of these data.

Crystalline Form IX of Pralsetinib may be further characterized by an X-ray powder diffraction pattern having peaks at 6.7, 9.7, 11.6, 13.4 and 17.0 degrees 2-theta±0.2 degrees 2-theta, and also having any one, two, three or four additional peaks selected from 5.5, 5.9, 17.5 and 20.2 degrees 2-theta±0.2 degrees 2-theta.

According to any aspect or embodiment of the present disclosure, crystalline Form IX of Pralsetinib may be alternatively characterized by an X-ray powder diffraction pattern having peaks at 5.5, 5.9, 6.7, 9.7, 11.6, 13.4, 17.0, 17.5, and 20.2 degrees 2-theta±0.2 degrees 2-theta.

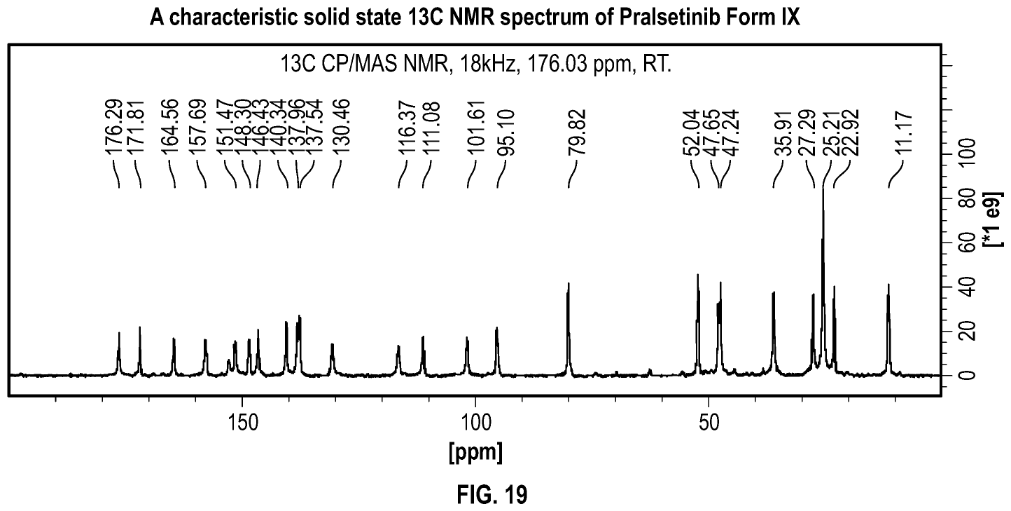

Alternatively or additionally, according to any aspect or embodiment of the present disclosure, Crystalline Form IX of Pralsetinib may be characterized by data selected from one or more of the following: a solid state ¹³C NMR having peaks at 157.7, 130.5, 35.9 and 25.2 ppm±0.2 ppm; or a solid state ¹³C NMR spectrum having chemical shift differences between a reference peak at 11.2±0.2 ppm of: 146.5, 119.3, 24.7 and 14.0±0.1 ppm respectively; or by a solid state ¹³C NMR spectrum substantially as depicted in FIG. 19; and combinations of these data.

In one embodiment of the present disclosure, crystalline Form IX of Pralsetinib is isolated.

Crystalline Form IX of Pralsetinib may be characterized by each of the above characteristics alone or by all possible combinations, e.g., an XRPD pattern having peaks at 6.7, 9.7, 11.6, 13.4 and 17.0 degrees 2-theta±0.2 degrees 2-theta; an XRPD pattern as depicted in FIG. 8, or a solid state ¹³C NMR spectrum substantially as depicted in FIG. 19 and any other combinations thereof.

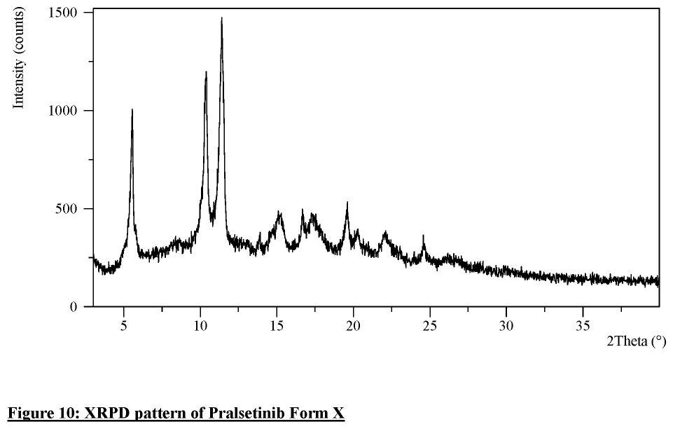

The present disclosure includes a crystalline polymorph of Pralsetinib, designated Form X. The crystalline Form X of Pralsetinib may be characterized by data selected from one or more of the following: an X-ray powder diffraction pattern substantially as depicted in FIG. 10; an X-ray powder diffraction pattern having peaks at 5.6, 10.4, 11.4, 15.1 and 19.6 degrees 2-theta±0.2 degrees 2-theta; and combinations of these data.

Crystalline Form X of Pralsetinib may be further characterized by an X-ray powder diffraction pattern having peaks at 5.6, 10.4, 11.4, 15.1 and 19.6 degrees 2-theta±0.2 degrees 2-theta, and also having any one, two, or three additional peaks selected from 16.7, 17.3 and 22.0 degrees 2-theta±0.2 degrees 2-theta.

According to any aspect or embodiment of the present disclosure, crystalline Form X of Pralsetinib may be alternatively characterized by an X-ray powder diffraction pattern having peaks at 5.6, 10.4, 11.4, 15.1, 16.7, 17.3, and 19.6 degrees 2-theta±0.2 degrees 2-theta.

In one embodiment of the present disclosure, crystalline Form X of Pralsetinib is isolated.

Crystalline Form X of Pralsetinib may be characterized by each of the above characteristics alone or by all possible combinations, e.g., an XRPD pattern having peaks at 10.4, 11.4, 15.1 and 19.6 degrees 2-theta±0.2 degrees 2-theta; an XRPD pattern as depicted in FIG. 10, and combinations thereof.

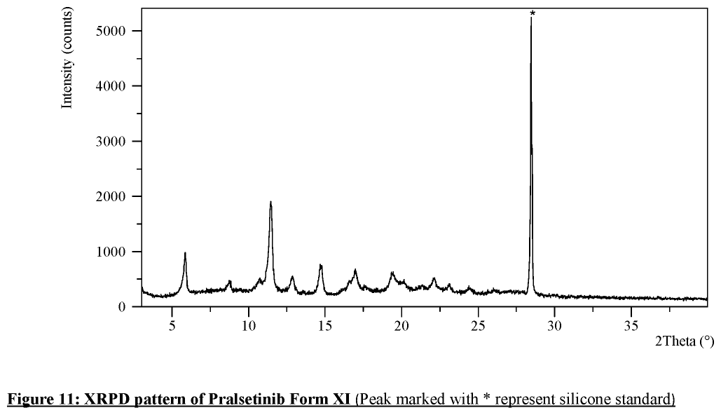

The present disclosure includes a crystalline polymorph of Pralsetinib, designated Form XI. The crystalline Form XI of Pralsetinib may be characterized by data selected from one or more of the following: an X-ray powder diffraction pattern substantially as depicted in FIG. 11; an X-ray powder diffraction pattern having peaks at 5.9, 8.8, 11.5, 14.8 and 17.0 degrees 2-theta±0.2 degrees 2-theta; and combinations of these data.

Crystalline Form XI of Pralsetinib may be further characterized by an X-ray powder diffraction pattern having peaks at 5.9, 8.8, 11.5, 14.8 and 17.0 degrees 2-theta±0.2 degrees 2-theta, and also having any one, two, or three additional peaks selected from 12.9, 19.4 and 22.1 degrees 2-theta±0.2 degrees 2-theta.

Thus, according to any aspect or embodiment of the present disclosure, crystalline Form XI of Pralsetinib may be characterized by: an X-ray powder diffraction pattern having peaks at 5.9, 8.8, 11.5, 12.9, 14.8 and 17.0 degrees 2-theta±0.2 degrees 2-theta; or an X-ray powder diffraction pattern having peaks at 5.9, 8.8, 11.5, 13.9, 14.8, 17.0 and 19.4 degrees 2-theta±0.2 degrees 2-theta; or an X-ray powder diffraction pattern having peaks at 5.9, 8.8, 11.5, 13.9, 14.8, 17.0 and 22.1 degrees 2-theta±0.2 degrees 2-theta.

According to any aspect or embodiment of the present disclosure, crystalline Form XI of Pralsetinib may be alternatively characterized by an X-ray powder diffraction pattern having peaks at 5.9, 8.8, 11.5, 12.9, 14.8, 17.0, 19.4, and 22.1 degrees 2-theta±0.2 degrees 2-theta.

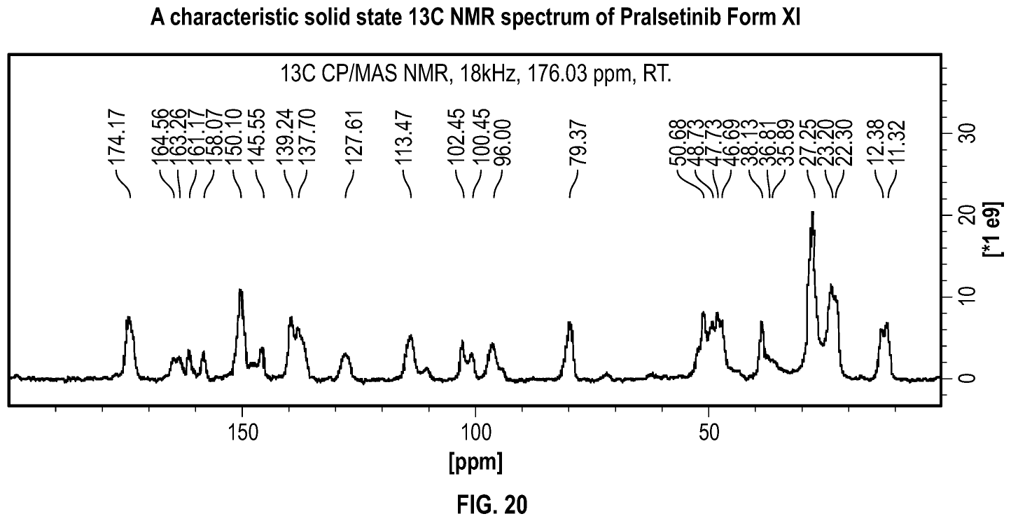

Alternatively or additionally, according to any aspect or embodiment of the present disclosure, crystalline Form XI of Pralsetinib may be characterized by data selected from one or more of the following: a solid state ¹³C NMR having peaks at 161.2, 158.1, 100.5 and 12.4 ppm±0.2 ppm; or a solid state ¹³C NMR spectrum having chemical shift differences between a reference peak at 79.4±0.2 ppm of: 81.8, 78.7, 21.1 and −67.0±0.1 ppm respectively; or by a solid state ¹³C NMR spectrum substantially as depicted in FIG. 20; and combinations of these data.

In one embodiment of the present disclosure, crystalline Form XI of Pralsetinib is isolated.

Crystalline Form XI of Pralsetinib may be characterized by each of the above characteristics alone or by all possible combinations, e.g., an XRPD pattern having peaks at 5.9, 8.8, 11.5, 14.8 and 17.0 degrees 2-theta±0.2 degrees 2-theta; an XRPD pattern as depicted in FIG. 11, or by a solid state ¹³C NMR spectrum substantially as depicted in FIG. 20, and any other combinations thereof.

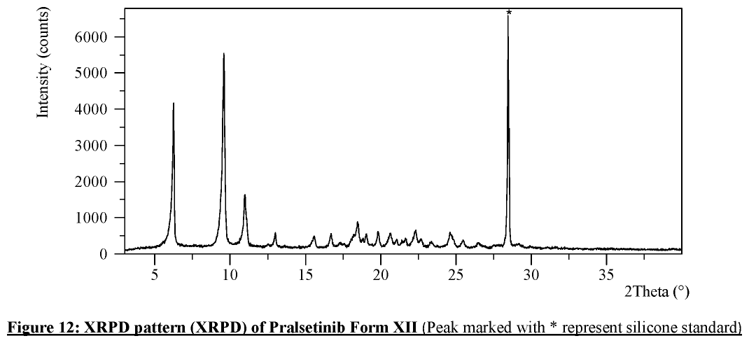

The present disclosure includes a crystalline polymorph of Pralsetinib, designated Form XII. The crystalline Form XII of Pralsetinib may be characterized by data selected from one or more of the following: an X-ray powder diffraction pattern substantially as depicted in FIG. 12; an X-ray powder diffraction pattern having peaks at 6.3, 9.6, 11.0, 13.0 and 19.8 degrees 2-theta±0.2 degrees 2-theta; and combinations of these data.

Crystalline Form XII of Pralsetinib may be further characterized by an X-ray powder diffraction pattern having peaks at 6.3, 9.6, 11.0, 13.0 and 19.8 degrees 2-theta±0.2 degrees 2-theta, and also having any one, two, three, four or five additional peaks selected from 15.6, 16.7, 18.4, 20.6 and 22.3 degrees 2-theta±0.2 degrees 2-theta.

According to any aspect or embodiment of the present disclosure, crystalline Form XII of Pralsetinib may be alternatively characterized by an X-ray powder diffraction pattern having peaks at 6.3, 9.6, 11.0, 13.0, 15.6, 16.7, 18.4, 19.8, 20.6, and 22.3 degrees 2-theta±0.2 degrees 2-theta.

In one embodiment of the present disclosure, crystalline Form XII of Pralsetinib is isolated.

Crystalline Form XII of Pralsetinib may be characterized by each of the above characteristics alone or by all possible combinations, e.g., an XRPD pattern having peaks at 6.3, 9.6, 11.0, 13.0 and 19.8 degrees 2-theta±0.2 degrees 2-theta; an XRPD pattern as depicted in FIG. 12, and combinations thereof.

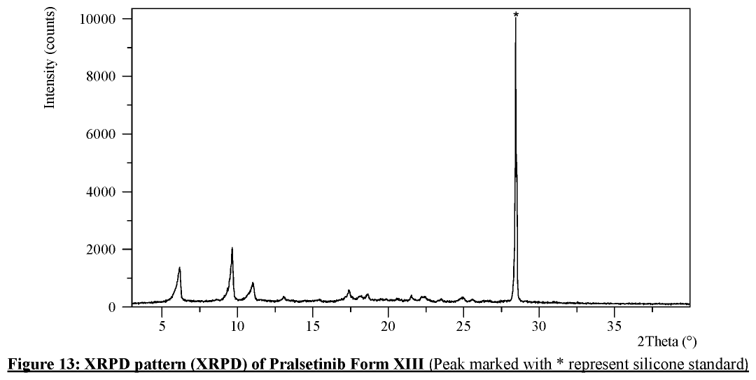

The present disclosure includes a crystalline polymorph of Pralsetinib, designated Form XIII The crystalline Form XIII of Pralsetinib may be characterized by data selected from one or more of the following: an X-ray powder diffraction pattern substantially as depicted in FIG. 13; an X-ray powder diffraction pattern having peaks at 6.2, 9.7, 11.1, 17.4 and 18.7 degrees 2-theta±0.2 degrees 2-theta; and combinations of these data.

In one embodiment of the present disclosure, crystalline Form XIII of Pralsetinib is isolated.

Crystalline Form XIII of Pralsetinib may be characterized by each of the above characteristics alone or by all possible combinations, e.g., an XRPD pattern having peaks at 6.2, 9.7, 11.1, 17.4 and 18.7 degrees 2-theta±0.2 degrees 2-theta; an XRPD pattern as depicted in FIG. 13, and combinations thereof.

In another aspect of the disclosure, there is provided Pralsetinib hydrochloride. According to any embodiment, Pralsetinib hydrochloride may be crystalline. The present disclosure further describes crystalline polymorphs of Pralsetinib hydrochloride.

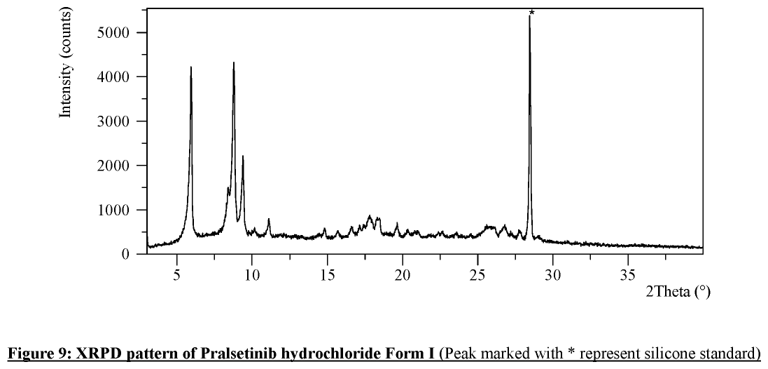

The present disclosure describes a crystalline polymorph of Pralsetinib hydrochloride, designated Form I. The crystalline Form I of Pralsetinib hydrochloride may be characterized by data selected from one or more of the following: an X-ray powder diffraction pattern substantially as depicted in FIG. 9; an X-ray powder diffraction pattern having peaks at 5.9, 8.8, 9.4, 11.1 and 19.6 degrees 2-theta±0.2 degrees 2-theta; and combinations of these data.

Crystalline Form I of Pralsetinib hydrochloride may be further characterized by an X-ray powder diffraction pattern having peaks at 5.9, 8.8, 9.4, 11.1 and 19.6 degrees 2-theta±0.2 degrees 2-theta, and also having any one, two, three or four additional peaks selected from 8.4, 14.8, 16.6, 17.8 and 18.3 degrees 2-theta±0.2 degrees 2-theta.

According to any aspect or embodiment of the present disclosure, crystalline Form I of Pralsetinib hydrochloride may be alternatively characterized by an X-ray powder diffraction pattern having peaks at 5.9, 8.4, 8.8, 9.4, 11.1, 14.8, 16.6, 17.8, 18.3, and 19.6 degrees 2-theta±degrees 2-theta.

In one embodiment of the present disclosure, crystalline Form I of Pralsetinib hydrochloride is isolated.

Crystalline Form I of Pralsetinib hydrochloride may be characterized by each of the above characteristics alone or by all possible combinations, e.g., an XRPD pattern having peaks at 5.9, 8.8, 9.4, 11.1 and 19.6 degrees 2-theta±0.2 degrees 2-theta; an XRPD pattern as depicted in FIG. 9, and combinations thereof.

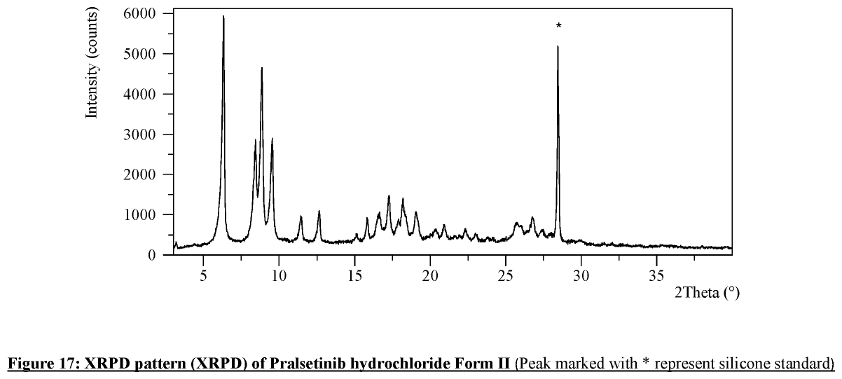

The present disclosure includes a crystalline polymorph of Pralsetinib hydrochloride, designated Form II. The crystalline Form II of Pralsetinib hydrochloride may be characterized by data selected from one or more of the following: an X-ray powder diffraction pattern substantially as depicted in FIG. 17; an X-ray powder diffraction pattern having peaks at 6.3, 11.5, 12.7, 17.3 and 19.0 degrees 2-theta±0.2 degrees 2-theta; and combinations of these data.

Crystalline Form II of Pralsetinib hydrochloride may be further characterized by an X-ray powder diffraction pattern having peaks at 6.3, 11.5, 12.7, 17.3 and 19.0 degrees 2-theta±degrees 2-theta, and also having any one or two additional peaks selected from: 3.2 and 23.0 degrees 2-theta±0.2 degrees 2-theta.

According to any aspect or embodiment of the present disclosure, crystalline Form II of Pralsetinib hydrochloride may be alternatively characterized by an X-ray powder diffraction pattern having peaks at 3.2, 6.3, 11.5, 12.7, 17.3, 19.0, and 23.0 degrees 2-theta±0.2 degrees 2-theta.

In one embodiment of the present disclosure, crystalline Form II of Pralsetinib hydrochloride is isolated.

Crystalline Form II of Pralsetinib hydrochloride may be characterized by each of the above characteristics alone or by all possible combinations, e.g., an XRPD pattern having peaks at 6.3, 11.5, 12.7, 17.3 and 19.0 degrees 2-theta±0.2 degrees 2-theta; an XRPD pattern as depicted in FIG. 17, and combinations thereof.

The present disclosure also encompasses salts of Pralsetinib and solid state forms thereof. Particularly, the present disclosure encompasses Pralsetinib L-(+)-tartrate and Pralsetinib tosylate.

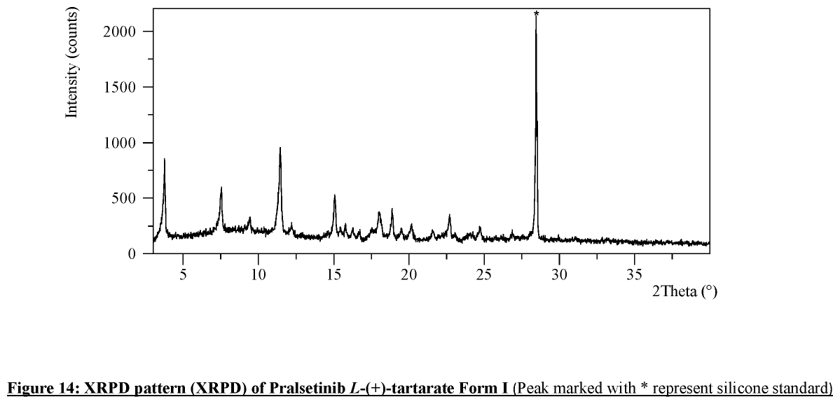

The present disclosure includes a crystalline polymorph of Pralsetinib L-(+)-tartrate, designated Form I. The crystalline Form I of Pralsetinib L-(+)-tartrate may be characterized by data selected from one or more of the following: an X-ray powder diffraction pattern substantially as depicted in FIG. 14; an X-ray powder diffraction pattern having peaks at 3.8, 7.5, 11.5, 15.1 and 18.9 degrees 2-theta±0.2 degrees 2-theta; and combinations of these data.

Crystalline Form I of Pralsetinib L-(+)-tartrate may be further characterized by an X-ray powder diffraction pattern having peaks at 3.8, 7.5, 11.5, 15.1 and 18.9 degrees 2-theta±0.2 degrees 2-theta, and also having any one, two, three, four or five additional peaks selected from 9.4, 12.2, 16.3, 18.0 and 20.2 degrees 2-theta±0.2 degrees 2-theta.

According to any aspect or embodiment of the present disclosure, crystalline Form I of Pralsetinib L-(+)-tartrate may be alternatively characterized by an X-ray powder diffraction pattern having peaks at 3.8, 7.5, 9.4, 11.5, 12.2, 15.1, 16.3, 18.0, 18.9, and 20.2 degrees 2-theta±0.2 degrees 2-theta.

In one embodiment of the present disclosure, crystalline Form I of Pralsetinib L-(+)-tartrate is isolated.

Crystalline Form I of Pralsetinib L-(+)-tartrate may be characterized by each of the above characteristics alone or by all possible combinations, e.g., an XRPD pattern having peaks at 3.8, 7.5, 11.5, 15.1 and 18.9 degrees 2-theta±0.2 degrees 2-theta; an XRPD pattern as depicted in FIG. 14, and combinations thereof.

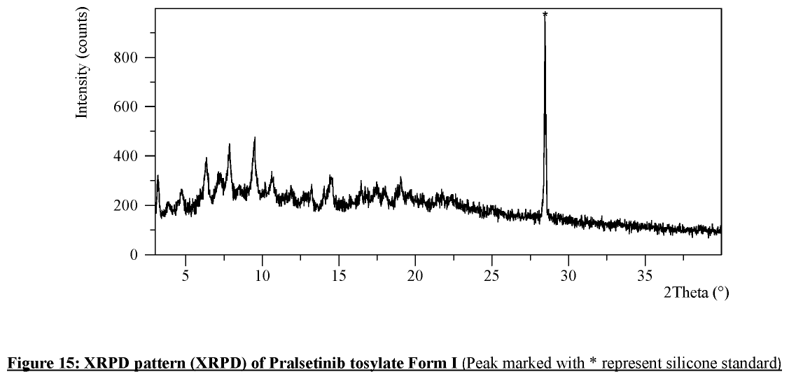

The present disclosure includes a crystalline polymorph of Pralsetinib tosylate, designated Form I. The crystalline Form I of Pralsetinib tosylate may be characterized by data selected from one or more of the following: an X-ray powder diffraction pattern substantially as depicted in FIG. 15; an X-ray powder diffraction pattern having peaks at 3.2, 6.3, 9.5, 10.7 and 14.5 degrees 2-theta±0.2 degrees 2-theta; and combinations of these data.

Crystalline Form I of Pralsetinib tosylate may be further characterized by an X-ray powder diffraction pattern having peaks at 3.2, 6.3, 9.5, 10.7 and 14.5 degrees 2-theta±0.2 degrees 2-theta, and also having any one or two additional peaks selected from 13.2 and 19.0 degrees 2-theta±0.2 degrees 2-theta.

According to any aspect or embodiment of the present disclosure, crystalline Form I of Pralsetinib tosylate may be alternatively characterized by an X-ray powder diffraction pattern having peaks at 3.2, 6.3, 9.5, 10.7, 13.2, 14.5, and 19.0 degrees 2-theta±0.2 degrees 2-theta.

In one embodiment of the present disclosure, crystalline Form I of Pralsetinib tosylate is isolated.

Crystalline Form I of Pralsetinib tosylate may be characterized by each of the above characteristics alone or by all possible combinations, e.g., an XRPD pattern having peaks at 3.2, 6.3, 9.5, 10.7 and 14.5 degrees 2-theta±0.2 degrees 2-theta; an XRPD pattern as depicted in FIG. 15, and combinations thereof.

The present disclosure further encompasses crystalline Pralsetinib: benzoic acid. Crystalline Pralsetinib: benzoic acid may be a co-crystal of Pralsetinib: benzoic acid.

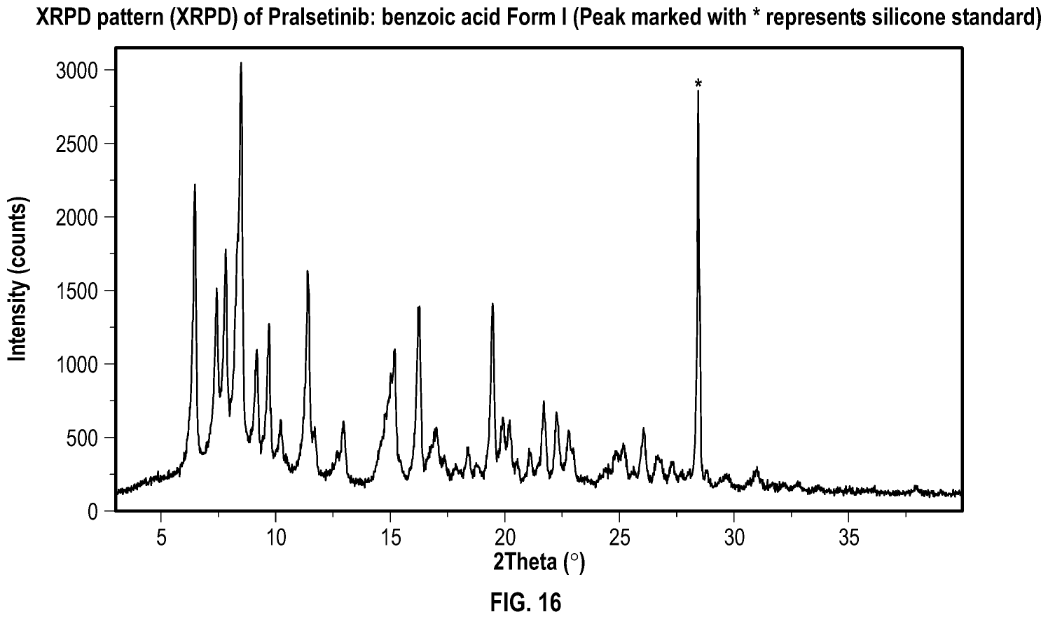

The disclosure further encompasses a crystalline form of Pralsetinib: benzoic acid, designated Form I. Crystalline Form I of Pralsetinib: benzoic acid may be characterized by data selected from one or more of the following: an X-ray powder diffraction pattern substantially as depicted in FIG. 16; an X-ray powder diffraction pattern having peaks at 6.5, 8.5, 11.4, 16.3 and 19.5 degrees 2-theta±0.2 degrees 2-theta; and combinations of these data.

Crystalline Form I of Pralsetinib: benzoic acid may be further characterized by an X-ray powder diffraction pattern having peaks at 6.5, 8.5, 11.4, 16.3 and 19.5 degrees 2-theta±0.2 degrees 2-theta, and also having any one, two, three, four or five additional peaks selected from 7.4, 7.8, 9.2, 13.0 and 15.2 degrees 2-theta±0.2 degrees 2-theta.

According to any aspect or embodiment of the present disclosure, crystalline Form I of Pralsetinib: benzoic acid may be alternatively characterized by an X-ray powder diffraction pattern having peaks at 6.5, 7.4, 7.8, 8.5, 9.2, 11.4, 13.0, 15.2, 16.3, and 19.5 degrees 2-theta±0.2 degrees 2-theta.

In embodiments of the present disclosure, crystalline Form I of Pralsetinib: benzoic acid is isolated.

Crystalline Form I of Pralsetinib: benzoic acid may be characterized by each of the above characteristics alone or by all possible combinations, e.g., an XRPD pattern having peaks at 6.5, 8.5, 11.4, 16.3 and 19.5 degrees 2-theta±0.2 degrees 2-theta; an XRPD pattern as depicted in FIG. 16; and combinations thereof.

In any aspect or embodiment of the present disclosure, any of the solid state forms of Pralsetinib, Pralsetinib salts or Pralsetinib cocrystals, described herein may be polymorphically pure or may be substantially free of any other solid state forms of the subject Pralsetinib, Pralsetinib salts or Pralsetinib cocrystals, respectively (for example a crystalline form of a Pralsetinib salt which is polymorphically pure, may be substantially free of any other solid state forms of the Pralsetinib salt; a crystalline form of Pralsetinib which is polymorphically pure, may be substantially free of any other solid state forms of the Pralsetinib; and likewise, a crystalline form of a Pralsetinib cocrystal which is polymorphically pure, may be substantially free of any other solid state forms of the Pralsetinib cocrystal). In any aspect or embodiment of the present disclosure, any of the solid state forms of Pralsetinib, Pralsetinib salts or Pralsetinib cocrystals described in any aspect or embodiment disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound (i.e. Pralsetinib, Pralsetinib salts or Pralsetinib cocrystals, respectively), preferably as measured by XRPD. Thus, any of the disclosed crystalline forms of Pralsetinib, Pralsetinib salts or Pralsetinib cocrystals, described herein may be substantially free of any other solid state forms of the subject Pralsetinib, Pralsetinib salts or Pralsetinib cocrystals respectively, and may contain greater than about 80% (w/w), greater than about 90% (w/w), greater than about 95% (w/w), greater than about 98% (w/w), greater than about 99% (w/w), or about 100% of the subject solid state form of the Pralsetinib, Pralsetinib salts or Pralsetinib cocrystals respectively.

The above crystalline polymorphs of Pralsetinib and of Pralsetinib salts and co-crystals can be used to prepare other crystalline polymorphs of Pralsetinib, Pralsetinib salts and/or co-crystals and their solid state forms.

The present disclosure encompasses a process for preparing other solid state forms of Pralsetinib, Pralsetinib salts and their solid state forms thereof.

The present disclosure provides the above described crystalline polymorphs of Pralsetinib and of Pralsetinib salts and co-crystals for use in the preparation of pharmaceutical compositions comprising Pralsetinib and/or crystalline polymorphs thereof.

The present disclosure also encompasses the use of crystalline polymorphs of Pralsetinib and of Pralsetinib salts and co-crystals of the present disclosure for the preparation of pharmaceutical compositions of crystalline polymorph Pralsetinib and/or crystalline polymorphs thereof.

The present disclosure includes processes for preparing the above mentioned pharmaceutical compositions. The processes include combining any one or a combination of the crystalline polymorphs of Pralsetinib or of Pralsetinib salts and co-crystals of the present disclosure with at least one pharmaceutically acceptable excipient.

Pharmaceutical combinations or formulations of the present disclosure contain any one or a combination of the solid state forms of Pralsetinib or of Pralsetinib salts and co-crystals of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Pralsetinib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Pralsetinib can be administered. Pralsetinib may be formulated for administration to a mammal, in embodiments to a human, by injection. Pralsetinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Pralsetinib and of Pralsetinib salts and co-crystals and the pharmaceutical compositions and/or formulations of Pralsetinib of the present disclosure can be used as medicaments, in embodiments in the treatment of non-small cell lung cancer (NSCLC) and thyroid cancer, or other RET-altered solid tumors.

The present disclosure also provides methods of treating non-small cell lung cancer (NSCLC) and thyroid cancer, or other RET-altered solid tumors by administering a therapeutically effective amount of any one or a combination of the crystalline polymorphs of Pralsetinib or of Pralsetinib salts and co-crystals of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 s, continuous scan. The described peak positions were determined using silicon powder as an internal standard in an admixture with the sample measured in case of Form II, Form III, Form IV, Form V, Form VI, Form VII, Form VIII, Form IX, Form XI, HCl forms I and II, L-tartrate Form I, tosylate Form I and Pralsetinib:benzoic acid Form I. The position of the silicon (Si) peak was corrected to silicone theoretical peak: 28.45° 2θ, and the positions of the measured peaks were corrected respectively. The described peak positions were determined without using silicon powder as an internal standard in case of Form X.

¹³C Solid State Nuclear Magnetic Resonance (“ss-NMR” or ¹³C Solid State NMR) Method

Solid-state NMR spectra were measured at 11.7 T using a Bruker Avance III HD 500 US/WB NMR spectrometer (Karlsruhe, Germany, 2013) with a 4- or 3.2-mm probehead.

The ¹³C CP/MAS NMR spectra employing cross-polarization were acquired using the standard cross-polarization pulse scheme at spinning frequency of 15 kHz. The cross-polarization contact time was 2 ms. The dipolar decoupling SPINAL64 was applied during the data acquisition. The number of scans was set for the signal-to-noise ratio SINO reach at least the value ca. 50. The ¹³C scale was referenced to α-glycine (176.03 ppm for ¹³C).

### EXAMPLES

### Preparation of Starting Materials

Pralsetinib can be prepared according to methods known from the literature, for example U.S. Pat. No. 10,030,005. For example, it can be prepared according to Example 5 or Example 9 therein.

### Example 1: Preparation of Pralsetinib Form II

50 mg of Pralsetinib was dissolved in 1.0 mL of 1,2-Propylene carbonate at temperature of about 150° C. Crystallization flask was sealed and left to crystallize at room temperature for period of about 1 day. The obtained solid was vacuum filtered over the black ribbon at room temperature and analyzed by XRPD. Pralsetinib Form II was obtained. An XRPD pattern is shown in FIG. 1.

### Example 2. Preparation of Pralsetinib Form III

50 mg of Pralsetinib was dissolved in 1.0 mL solvent mixture of 2-Propanol and water (ratio 1:1) at temperature of about 80° C. Crystallization flask was sealed and left to crystallize at room temperature for 1 day. The obtained solid was vacuum filtered over the black ribbon at room temperature and analyzed by XRPD. Pralsetinib Form III was obtained. An XRPD pattern is shown in FIG. 2.

### Example 3. Preparation of Pralsetinib Form V

50 mg of Pralsetinib base was dissolved in 4.0 mL solvent mixture of Methanol and water (ratio 1:1) at temperature of about 60° C. Crystallization flask was sealed and left to crystallize at room temperature for a period of about 1 day. The obtained solid was vacuum filtered over the black ribbon at room temperature and analyzed by XRPD. Pralsetinib Form V was obtained. An XRPD pattern is shown in FIG. 3.

### Example 4. Preparation of Pralsetinib Form VI

50 mg of Pralsetinib was dissolved in 1.0 mL of Ethylene glycol at temperature of about 150° C. Crystallization flask was sealed and left to crystallize at room temperature for period of about 1 day. The obtained solid was vacuum filtered over the black ribbon at room temperature and analyzed by XRPD. Pralsetinib Form VI was obtained. An XRPD pattern is shown in FIG. 4.

### Example 5. Preparation of Pralsetinib Form IV

300 mg of Pralsetinib Form III was slurried in 9.0 mL of iso-Butyl acetate at a temperature of about 110° C. for period of about 2 hours. The obtained solid was vacuum filtered over the black ribbon at room temperature and analyzed by XRPD. Pralsetinib Form IV was obtained. An XRPD pattern is shown in FIG. 5.

### Example 6. Preparation of Pralsetinib Form VII

300 mg of Pralsetinib was dissolved in 8.0 mL of Methanol, stirred and sonicated on ultrasonic bath at room temperature for period of about 30 seconds. The solution was filtered over the black ribbon at room temperature and slightly heated at a temperature of about 40° C. Aliquot of 0.350 mL of solution was pipetted in crystallization flask and 1.05 mL of Hexane was added drop-by-drop at room temperature. Crystallization flask was closed and left to crystallize at room temperature for 7 days. The obtained solid was vacuum filtered over the black ribbon at room temperature and analyzed by XRPD. Pralsetinib Form VII was obtained. An XRPD pattern is shown in FIG. 6.

### Example 7. Preparation of Pralsetinib Form VIII

200 mg of Pralsetinib was dissolved in 4.5 mL of Methanol, stirred, sonicated on ultrasonic bath and heated to temperature of about 40° C.° C. The solution was filtered over the black ribbon at room temperature. Aliquot of 0.450 mL of solution was pipetted in crystallization flask and 1.5 mL of Toluene was added drop-by-drop at room temperature. Crystallization flask was closed and left to crystallize in refrigerator at temperature of about 5° C. The obtained solid was vacuum filtered over the black ribbon at room temperature and analyzed by XRPD. Pralsetinib Form VIII was obtained. An XRPD pattern is shown in FIG. 7.

### Example 8. Preparation of Pralsetinib Form IX

300 mg of Pralsetinib Form III was slurried in 12.0 mL of Heptane at a temperature of about 95° C. for period of about 1 day. The obtained solid was vacuum filtered over the black ribbon at room temperature and analyzed by XRPD. Pralsetinib Form IX was obtained. An XRPD pattern is shown in FIG. 8.

### Example 9. Preparation of Pralsetinib Hydrochloride Form I

Pralsetinib base (1920 mg, Form II) was dissolved in methanol (24 mL) at room temperature. Hydrochloric acid (4.0 mL, 1.0M) was added dropwise and a solution was formed (molar ratio of Pralsetinib and hydrochloric acid was 1:1.1). iso-butyl acetate (25 mL) was added dropwise to the solution, and a suspension was obtained. The suspension was stirred at room temperature for 16 hours and vacuum filtered over the black ribbon at room temperature. The isolated solid was analyzed by XRPD, Pralsetinib hydrochloride Form I was obtained. An XRPD pattern is shown in FIG. 9.

### Example 10. Preparation of Pralsetinib Form X

2 mg of Pralsetinib Form VII was placed in pin hole aluminum pan. Sample was subjected to thermal treatment in DSC Discovery TA Instruments, according to steps of heating of the sample by heating rate of about 10° C./minute up to temperature of about 110° C., isothermal heating for period of about 10 minutes at temperature of about 110° C., and cooled to room temperature. The obtained solid was analyzed by XRPD. Pralsetinib Form X was obtained. An XRPD pattern is shown in FIG. 10.

### Example 11. Preparation of Pralsetinib Form XI

5.45 mg of Pralsetinib Form III was placed in open aluminum pan. Sample was subjected to thermal treatment in TGA Discovery TA Instruments, according to steps of heating of the sample by heating rate of about 10° C./minute up to temperature of 120° C., isothermal heating for period of about 30 minutes at temperature of about 120° C., and cooled to room temperature. The obtained solid was analyzed by XRPD. Pralsetinib Form XI was obtained An XRPD pattern is shown in FIG. 11.

### Example 12. Preparation of Pralsetinib Form XII

Pralsetinib base (20 mg, Form X) was placed in Eppendorf epruvete, which was put in a crystallization flask with methanol (1 mL). After 1 day, the sample was analyzed by XRPD, Pralsetinib base Form XII was obtained. An XRPD pattern is shown in FIG. 12.

### Example 13. Preparation of Pralsetinib Form XIII

Pralsetinib base (Form VII, 50 mg) was placed in an open Petrium dish and then kept at conditions with 20% relative humidity at room temperature. After 7 days, the sample was analyzed by XRPD, Pralsetinib base Form XIII was obtained. An XRPD pattern is shown in FIG. 13.

### Example 14. Preparation of Pralsetinib Hydrochloride Form II

Pralsetinib hydrochloride (200 mg, Form I) was heated in a vacuum oven at 80° C. at a pressure of 10 mbar for a period of about 8 hours. The obtained solid was analyzed by XRPD, Pralsetinib hydrochloride Form II was obtained. An XRPD pattern is shown in FIG. 17.

### Example 15. Preparation of Pralsetinib L-(+)-Tartrate Form I

Pralsetinib base (936.6 mg, Form II) and L-(+)-Tartaric acid (263.4 mg) (molar ratio of Pralsetinib and L-(+)-Tartaric acid was 1:1) were charged and acetone (20 mL) was added. The obtained suspension was stirred at room temperature for 3 days. Then, the solid was vacuum filtered over the black ribbon at room temperature. The isolated solid was analyzed by XRPD, Pralsetinib L-(+)-tartrate Form I was obtained. An XRPD pattern is shown in FIG. 14.

### Example 16. Preparation of Pralsetinib Tosylate Form I

Pralsetinib (250 mg, Form II) and p-toluenesulfonic acid (98 mg, monohydrate) (molar ratio of Pralsetinib and L-(+)-Tartaric acid was 1:1.1) were dissolved in a mixture of methanol and water (10 mL MeOH:Water 2:1). Into the obtained solution, i-BuOAc (30 mL) was added dropwise. The obtained suspension was stirred at room temperature for 16 hours and vacuum filtered over the black ribbon at room temperature. The isolated solid was analyzed by XRPD. An XRPD pattern is shown in FIG. 15.

### Example 17. Preparation of Pralsetinib: Benzoic Acid Form I (Co-Crystal)

Pralsetinib base (399.4 mg, Form II) and Benzoic acid (100.6 mg) (molar ratio of Pralsetinib and L-(+)-Tartaric acid was 1:1.1) were charged and water (25 mL) was added. The obtained suspension was stirred at 40° C. for 1 day, then it was vacuum filtered over the blue ribbon at room temperature. The isolated solid was analyzed by XRPD, a Co-crystal of Pralsetinib base and benzoic acid Form I was obtained. An XRPD pattern is shown in FIG. 16.

### Example 18. Preparation of Pralsetinib Hydrochloride Form II

Into 50 mL one neck flask equipped with magnetic stirrer bar and condenser were loaded: Sodium (1S,4S)-1-methoxy-4-(4-methyl-6-((5-methyl-1H-pyrazol-3-yl)amino)pyrimidin-2-yl)cyclohexane-1-carboxylate sodium (1.0 gram), (5)-1-(6-(4-fluoro-1H-pyrazol-1-yl)pyridin-3-yl)ethan-1-amine hydrochloride (660 mg) and Acetonitrile (“ACN”, 20 mL) followed by Triethylamine (“TEA”, 1.14 mL), an activating agent (ethyl cyanohydroxyiminoacetate—“Oxyma Pure”, 271 mg) and 1-(3-Dimethylaminopropyl)-3-ethylcarbodiimide hydrochloride (EDC×HCL) (939.3 mg). The reaction mixture was cooled down to about room temperature. NaCl, (10% aq solution, 20 mL) was added, the layers were separated and the organic layer was additionally washed with NaCl (10% aq solution, 20 mL). The layers were separated, the organic layer was heated to 40° C. and 1.0 M HCL (˜8.0 mL) was added. The obtained thick suspension was heated to a temperature of about 60° C. and was stirred at 60° C. for about 1 hour. Then, the suspension was allowed to cool to about room temperature. The suspension was maintained at room temperature overnight. Water (14 mL) was added in a dropwise manner to the suspension at room temperature, and the suspension was left stirring at room temperature for 1 hour. The formed crystals were filtered off and were washed with a mixture of acetonitrile and water (ACN/H2O, 1:1) (2×6 mL) and then dried in a vacuum oven at a temperature of about 50° C. and pressure of about 10 mbar for about 8 hours. The obtained solid was analyzed by XRPD, Pralsetinib hydrochloride Form II was obtained.

### Example 19. Preparation of Pralsetinib Form V

Pralsetinib HCl (3.0 grams) was charged into a three necked flask, MeOH (51.6 ml, 17.2 V) was added and the obtained reaction mixture was heated to 50° C. The pH of the reaction mixture was set to 7.7 with 6M NaOH. The obtained solution was filtered through a red ribbon filter paper. The filtrate was charged into 250 ml reactor. The filter flask was washed with MeOH (9.0 ml, 3 V), and the wash was added to the filtrate in the reactor. The yellow solution was heated to 50° C., and water (37 ml, 12.3 V) was added dropwise. The reaction mixture was slowly cooled (using a thermostat, cooling speed 0.4° C./min) to a temperature of 31° C. to 32° C., and at that temperature the solution was seeded with Pralsetinib form V (1% w/w, relative to the starting material Pralsetinib HCl). The obtained reaction mixture was cooled to a temperature of about 0-5° C. (using a thermostat, cooling speed 0.4° C./min). The obtained suspension was stirred for 1 hour at a temperature of about 0 to 5° C. The obtained crystals were filtered off, washed with a pre-cooled mixture of methanol and water (MeOH/water=1/4, 2×3 V, precooled to 5-10° C.) and then it was washed with water (5 V). The crystals were dried in a vacuum oven at a temperature of about 50° C. and pressure of about 10 mbar for about 8 hours. The obtained solid was analyzed by XRPD. Pralsetinib Form V was obtained. An XRPD pattern is shown in FIG. 21.

### Example 20. Preparation of Pralsetinib Form V

Pralsetinib HCl (3.0 grams) was charged into a three necked flask, MeOH (51.6 ml, 17.2 V) was added and the pH of the obtained reaction mixture was set to 7.6 with 6M NaOH. The obtained yellow solution was filtered through a red ribbon filter paper. The filtrate was charged into a 250 ml reactor. The filter flask and the three necked flask were washed with MeOH (each with 4.5 ml, 1.5 V), and the wash was added to the filtrate in the reactor. The yellow solution was heated to a temperature of about 50° C., and water (37 ml, 12.3 V) was added dropwise. The obtained reaction mixture was slowly cooled down (using a thermostat, cooling speed 0.4° C./min) to 31° C.-32° C., and at that temperature the solution was seeded (1% w/w, relative to the starting material Pralsetinib HCl) with Pralsetinib form V. The obtained reaction mixture was cooled down to 0-5° C. (using a thermostat, cooling speed 0.4° C./min). At a temperature of about 0-5° C. water (22.8 ml, 7.6 V) was added dropwise. The obtained suspension was stirred for 1 hour at a temperature of about 0-5° C. The obtained crystals were filtered off, washed with a precooled mixture of methanol and water (MeOH/water=1/4) (2×3 V, precooled to 5-10° C.) and then it was washed with water (5 V). The crystals were dried in a vacuum oven at a temperature of about 50° C. and pressure of about 10 mbar for about 8 hours. The obtained solid was analyzed by XRPD. Pralsetinib Form V was obtained.

### Example 21. Preparation of Pralsetinib Form XI

About 600 mg of Pralsetinib form III was heated in vacuum oven at 120° C. for 3 hours. Sample was analyzed by XRPD. Pralsetinib form XI was obtained.

1. Crystalline Form V of Pralsetinib characterized by data selected from:
a) an X-ray powder diffraction pattern substantially as depicted in FIG. 3;
b) an X-ray powder diffraction pattern substantially as depicted in FIG. 21;
c) an X-ray powder diffraction pattern having peaks at 11.2, 11.7, 13.2, 15.1 and 17.2 degrees 2-theta±0.2 degrees 2-theta;
d) a solid state ¹³C NMR spectrum having peaks at 172.2, 160.3, 103.1 and 38.3 ppm±0.2 ppm;
e) a solid state ¹³C NMR spectrum having chemical shift differences between a reference peak at 11.7±0.2 ppm of: 160.5, 148.6, 91.3 and 26.6±0.1 ppm respectively;
f) a solid state ¹³C NMR substantially as depicted in FIG. 18; and
g) any combination of (a)-(f).

2. Crystalline Form V of Pralsetinib according to claim 1, which is characterized by an X-ray powder diffraction pattern having peaks at 11.2, 11.7, 13.2, and 17.2 degrees 2-theta±0.2 degrees 2-theta, and also having any one, two, three, four or five additional peaks selected from 14.1, 19.7, 20.9, 22.4 and 23.5 degrees 2-theta±degrees 2-theta.

3. Crystalline Form V of Pralsetinib according to claim 1, which is characterized by an X-ray powder diffraction pattern having peaks at 11.2, 11.7, 13.2, 14.1, 15.1, 17.2, 19.7, 20.9, 22.4 and 23.5 degrees 2-theta±0.2 degrees 2-theta.

4. Crystalline Form V of Pralsetinib according to claim 1, which is further characterized by an X-ray powder diffraction pattern having an absence of peaks at 6.2 to 8.1 degrees 2-theta±0.2 degrees 2-theta.

5. Crystalline Form V of Pralsetinib according to claim 1 which is further characterized by an X-ray powder diffraction pattern having an absence of peaks at 9.0 to 10.2 degrees 2-theta±0.2 degrees 2-theta.

6. Crystalline Form V of Pralsetinib according to claim 1, which is further characterized by an X-ray powder diffraction pattern having an absence of peaks at 6.2 to 8.1 degrees 2-theta and an absence of peaks at 9.0 to degrees 2-theta±0.2 degrees 2-theta.

7. Crystalline Form V of Pralsetinib according to claim 1, which is characterized by the following unit cell data:

| Unit cell parameter | Le Bail |
|---|---|
| cell_length_a | 8.3649(8) Å |
| cell_length_b | 5.6382 (7) Å |
| cell_length_c | 30.9041 (4) Å |
| cell_angle_alpha | 90° |
| cell_angle_beta | 93.644(6)° |
| cell_angle_gamma | 90° |
| cell_volume | 1454.6 (3) Å³ |
| symmetry_cell_setting | Monoclinic |
| Symmetry space group name | P2₁ |

8. Crystalline Form V of Pralsetinib according to claim 1 which contains no more than about 20% of any other crystalline forms of Pralsetinib.

9. Crystalline Form V of Pralsetinib according to claim 1 which contains no more than about 20% of amorphous Pralsetinib.

10. Crystalline Form IX of Pralsetinib characterized by data selected from:
a) an X-ray powder diffraction pattern substantially as depicted in FIG. 8;
b) an X-ray powder diffraction pattern having peaks at 6.7, 9.7, 11.6, 13.4 and 17.0 degrees 2-theta±0.2 degrees 2-theta;
c) a solid state ¹³C NMR spectrum having peaks at 157.7, 130.5, 35.9 and 25.2 ppm±0.2 ppm;
d) a solid state ¹³C NMR spectrum having chemical shift differences between a reference peak at 11.2±0.2 ppm of: 146.5, 119.3, 24.7 and 14.0±0.1 ppm respectively;
e) solid state ¹³C NMR substantially as depicted in FIG. 19; and
f) any combination of (a)-(e).

11. Crystalline Form IX of Pralsetinib according to claim 10, which is characterized by an X-ray powder diffraction pattern having peaks at 6.7, 9.7, 11.6, 13.4 and 17.0 degrees 2-theta±0.2 degrees 2-theta, and also having any one, two, three or four additional peaks selected from 5.5, 5.9, 17.5 and 20.2 degrees 2-theta±0.2 degrees 2-theta.

12. Crystalline Form IX of Pralsetinib according to claim 10, which is characterized by an X-ray powder diffraction pattern having peaks at 5.5, 5.9, 6.7, 9.7, 11.6, 13.4, 17.0, 17.5 and 20.2 degrees 2-theta±0.2 degrees 2-theta.

13. Crystalline Form IX of Pralsetinib according to claim 10, which contains no more than about 20% of any other crystalline forms of Pralsetinib.

14. Crystalline Form IX of Pralsetinib according to claim 10, which contains no more than about 20% of amorphous Pralsetinib.

15. Crystalline Form XI of Pralsetinib characterized by data selected from:
a) an X-ray powder diffraction pattern substantially as depicted in FIG. 11;
b) an X-ray powder diffraction pattern having peaks at 5.9, 8.8, 11.5, 14.8 and 17.0 degrees 2-theta±0.2 degrees 2-theta;
c) a solid state ¹³C NMR spectrum having peaks at 161.2, 158.1, 100.5 and 12.4 ppm±0.2 ppm;
d) a solid state ¹³C NMR spectrum having chemical shift differences between a reference peak at 79.4±0.2 ppm of: 81.8, 78.7, 21.1 and −67.0±0.1 ppm respectively;
e) solid state ¹³C NMR substantially as depicted in FIG. 20; and
f) any combination of (a)-(e).

16. Crystalline Form XI of Pralsetinib according to claim 15, which is characterized by an X-ray powder diffraction pattern having peaks at 5.9, 8.8, 11.5, 14.8 and 17.0 degrees 2-theta±0.2 degrees 2-theta, and also having any one, two, or three additional peaks selected from 12.9, 19.4 and 22.1 degrees 2-theta±0.2 degrees 2-theta.

17. Crystalline Form XI of Pralsetinib according to claim 16, which is characterized by having an X-ray powder diffraction pattern having an additional peak at 12.9 degrees 2-theta±0.2 degrees 2-theta.

18. Crystalline Form XI of Pralsetinib according to claim 16, which is characterized by having an X-ray powder diffraction pattern having an additional peak at 19.4 degrees 2-theta±0.2 degrees 2-theta.

19. Crystalline Form XI of Pralsetinib according to claim 16, which is characterized by having an X-ray powder diffraction pattern having an additional peak at 22.1 degrees 2-theta±0.2 degrees 2-theta.

20. Crystalline Form XI of Pralsetinib according to claim 15, which is characterized by an X-ray powder diffraction pattern having peaks at 5.9, 8.8, 11.5, 12.9, 14.8, 17.0, 19.4 and 22.1 degrees 2-theta±0.2 degrees 2-theta.

21. Crystalline Form XI of Pralsetinib according to claim 15, which contains no more than about 20% of any other crystalline forms of Pralsetinib.

22. Crystalline Form XI of Pralsetinib according to claim 15, which contains no more than about 20% of amorphous Pralsetinib.

23. A pharmaceutical composition comprising a crystalline form of Pralsetinib according to claim 1.

24. A pharmaceutical formulation comprising a crystalline form of Pralsetinib according to claim 1, and at least one pharmaceutically acceptable excipient.

25. A process for preparing a pharmaceutical formulation comprising combining a crystalline form of Pralsetinib according to claim 1 with at least one pharmaceutically acceptable excipient.

26. (canceled)

27. A medicament comprising the crystalline form of Pralsetinib according to claim 1.

28. (canceled)

29. A method of treating cancer comprising administering a therapeutically effective amount of a crystalline form of Pralsetinib according to claim 1, to a subject in need of the treatment.

30. (canceled)

31. (canceled)

32. A process for preparing solid state form of Pralsetinib, Pralsetinib salts, co-crystal or solid state form thereof, comprising preparing a crystalline form of Pralsetinib according to claim 1, and converting it to another solid state form of Pralsetinib, Pralsetinib salts, co-crystal or solid state form thereof.
